(19) United States
(12) Patent Application Publication
(10) Pub. No.: US 2026/0148875 A1
(43) Pub. Date: May 28, 2026

(54) SLICE-SHAPED SCATTERED RADIATION MASK

(71) Applicant: Siemens Healthineers AG, Forchheim (DE)

(72) Inventors: Martin BRAEUER, Nuernberg (DE); Klaus-Peter KREUZER, Fuerth (DE)

(73) Assignee: Siemens Healthineers AG, Forchheim (DE)

(21) Appl. No.: 19/396,994

(22) Filed: Nov. 21, 2025

(30) Foreign Application Priority Data
Nov. 22, 2024 (EP) 24214811.2
Dec. 23, 2024 (EP) 24222839.3
Mar. 25, 2025 (EP) 25166000.7

(51) Int. Cl. G21K 1/12; A61B 6/58; B22F 12/30; B22F 12/53; B22F 12/55; B29C 64/209; B29C 64/245; B29C 64/336; B29K 105/00; B29L 31/00; B33Y 10/00; B33Y 30/00; B33Y 80/00

(52) U.S. Cl. CPC G21K 1/12; A61B 6/58; B22F 12/30; B22F 12/53; B22F 12/55; B29C 64/209; B29C 64/245; B29C 64/336; B33Y 10/00; B33Y 30/00; B33Y 80/00; B22F 2998/10; B29K 2105/0005; B29L 2031/753

(57) ABSTRACT

A scattered radiation mask for reduction of scattered x-ray radiation, the scattered radiation mask comprising: a stack of slices, wherein the stack of slices has an x-ray radiation-transparent first slice, an x-ray radiation-absorbing second slice and an x-ray radiation-transparent third slice. The first slice, the second slice and the third slice each have at least one planar layer, and neighboring layers of the stack of slices are applied to each other in a planar manner and in a stack direction via an additive manufacturing method.

### CROSS-REFERENCE TO RELATED APPLICATION(S)

The present application claims priority under 35 U.S.C. § 119 to European Patent Application No. 24214811.2, filed Nov. 22, 2024, European Patent Application No. 24222839.3, filed Dec. 23, 2024, and European Patent Application No. 25166000.7, filed Mar. 25, 2025, the entire contents of each of which are incorporated herein by reference.

### FIELD

One or more example embodiments of the present invention relate to a scattered radiation mask, to an arrangement for an x-ray imaging with the scattered radiation mask, to a method for manufacturing a scattered radiation mask for reduction of scattered x-ray radiation, to a system for additive manufacturing of a scattered radiation mask for reduction of scattered x-ray radiation and to the use of a system with an application unit for additive manufacturing of a scattered radiation mask for reduction of scattered x-ray radiation.

One or more example embodiments of the present invention also relate to the field of in particular medical x-ray imaging, in particular to scattered radiation masks for improving the image quality in x-ray recordings.

### BACKGROUND

In modern x-ray diagnostics reducing scattered x-ray radiation is of great importance in order to obtain high-contrast and highly detailed recordings. Conventional scattered radiation masks in strip form regularly consist of lead lamellae, which are arranged between x-ray radiation-transparent materials, for example paper strips. This technology has limitations however in respect of the resolution and flexibility able to be achieved in manufacturing.

As shown in FIGS. 1 and 2, a detector for x-ray radiation is struck not only by (possibly attenuated) x-ray photons, so-called primary x-ray radiation, but also by scattered x-ray photons (scattered x-ray radiation). This scattering process has physical reasons and in principle cannot be avoided. This so-called scattered x-ray radiation arises in any material struck by the x-ray radiation, in particular as a function of its nature. Also, and in particular aqueous materials (like the human body) generate strong scattered x-ray radiation. In particular diagnostic x-ray images of humans in medicine can become unclear due to the scattered x-ray photons, since the sharp depiction of anatomical details is overlaid by the diffuse scattered x-ray radiation. For good depictions of fine details x-ray imaging systems, in particular those for medical engineering, must efficiently suppress scattered x-ray radiation.

A widely-used technique for suppression of scattered x-ray radiation is the use of a scattered radiation mask in the form of a grid or a strip. The x-ray radiation-absorbing structures consist of a material with a high x-ray radiation attenuation characteristic. This ensures that only x-ray photons can reach the x-ray detector via the direct line of sight from the x-ray radiation source (assumed to be punctiform), while scattered x-ray photons will be absorbed due to the angle being too acute.

The demands made on scattered radiation masks, in particular for medical x-ray imaging systems, are high, since on the one hand the scattered x-ray photons are to be absorbed as completely as possible, on the other hand the primary x-ray radiation is to reach the x-ray detector as unimpeded as possible. A further important requirement is that the scattered radiation mask is ideally not visible in the resulting x-ray image, although areas of the x-ray detector are covered by the material of the scattered radiation mask, in particular by the x-ray radiation-absorbing slices, and thus these covered areas cannot be reached directly by x-ray photons from the x-ray radiation source. For this reason the amount of material of the x-ray radiation-absorbing slices should be reduced to an absolute minimum. In the ideal case a scattered radiation mask thus consists of thin metal strips as x-ray radiation-absorbing slices, which for their part are just thick enough to absorb the scattered radiation. Typically fine strips of lead, of for example 20 μm thickness, are employed. For example strip widths of approx. 3 mm at a spacing of appr. 100 μm have proven themselves in practice for very high-quality x-ray images.

Lead strips with cross sections of 0.020 mm×3 mm and typical lengths of 400 mm are however mechanically completely unstable. For this reason a filler material is introduced between these x-ray radiation-absorbing slices. This filler material is undesirable for the process of x-ray imaging, since typically it absorbs x-ray radiation at least to a small extent, i.e. it is not entirely x-ray radiation-transparent. Ultimately all absorption of x-ray photons from the body leads to the need to increase the potentially harmful radiation dose employed for the examination. An important development goal for medical x-ray devices is therefore the minimizing of the amount of material between the patients and the x-ray detector.

In the medical engineering market lightweight metals, such as aluminum for example, can be found as a filler between the x-ray radiation-absorbing slices. These solutions however have the disadvantage that such a filler material has a comparatively high x-ray radiation attenuation characteristic. Technically stacks of slices with for example 20 μm lead strips, which are alternated with for example 100 μm thick aluminum strips and glued, can be produced relatively easily and efficiently.

Known internally is another way for x-ray radiation-transparent filler material: Instead of aluminum as the filler material for the x-ray radiation-transparent slices, the filler material paper is employed. The paper-lead strips are glued together. The paper-adhesive combination, with major chemical elements hydrogen, carbon and oxygen, has a far lower x-ray absorption than the aluminum used otherwise. A substitute for the method described in which the poisonous lead and the natural product paper can be dispensed with is not currently known. The material lead, that is currently used as an absorber, is increasingly problematic, since its use, on account of the poisonous nature of lead, is to be reduced as far as possible, or it is likely in the future that the use of lead as a material will be banned. The use of paper as a filler is technically challenging however: Paper is a natural product, of which an even quality can only be ensured at great expense. Paper is hygroscopic, which is why the layering and processing must be undertaken in an exact temperature and humidity-controlled environment. The compatibility of the adhesive systems used is critical. Since the adhesion of the adhesive/paper slices is critical, the durability of the finished plates is much less than with other methods. In order to stabilize these scattered radiation masks and seal them, these are typically glued in between additional (absorbing) plates made of filler material.

Current systems for scattered radiation reduction thus exhibit a number of technical limitations. The manufacturing of scattered radiation masks with very fine structures is difficult and costly with conventional methods. Moreover, the materials used are often not optimally tailored to the specific requirements of various applications of x-ray imaging. The lack of flexibility in the design of the scattered radiation masks leads to compromises between scattered radiation reduction and primary radiation transmission. What is more, the integration of scattered radiation masks into modern digital x-ray systems represents a challenge, in particular when it is a matter of adapting them to various imaging geometries.

### SUMMARY

An underlying object of one or more example embodiments of the present invention is to specify a scattered radiation mask, an arrangement for x-ray imaging with the scattered radiation mask, a method for manufacturing a scattered radiation mask for reduction of scattered x-ray radiation, a system for additive manufacturing of a scattered radiation mask for reduction of scattered x-ray radiation and the use of a system with an application unit for additive manufacturing of a scattered radiation mask for reduction of scattered x-ray radiation, which are improved, preferably which make possible an improved scattered radiation reduction with simultaneous high primary radiation transmission, are flexible in their manufacture and adaptation to various x-ray applications and/or are able to be integrated easily into modern x-ray imaging systems.

At least the above-mentioned object is achieved by the features of the independent claims. Advantageous embodiments are described in the dependent claims.

According to one or more example embodiments of the present invention, the inventive scattered radiation mask for reduction of scattered x-ray radiation, in particular for x-ray imaging, has
a stack of slices,
wherein the stack of slices has an x-ray radiation-transparent first slice, an x-ray radiation-absorbing second slice and an x-ray radiation-transparent third slice,
wherein the first slice, the second slice and the third slice each have at least one planar layer,
wherein neighboring layers of the stack of slices are applied onto one another in a planar manner in the stack direction via an additive manufacturing method.

According to one or more example embodiments of the present invention, an inventive method for manufacturing of a scattered radiation mask for reduction of scattered x-ray radiation, in particular for x-ray imaging, comprises the steps:
Application via an additive manufacturing method of layers in a planar manner onto one another in the stack direction to form a stack of slices in such a way,
that the stack of slices has an x-ray radiation-transparent first slice, an x-ray radiation-absorbing second slice and an x-ray radiation-transparent third slice and
that the first slice, the second slice and the third slice each have at least one planar layer.

An inventive step of one or more example embodiments of the present invention includes an innovative way of looking at the direction of the additive manufacturing. This is because the layers of the x-ray radiation-transparent and x-ray radiation-absorbing slices are applied to one another in a planar manner in the stack direction. The scattered radiation mask is thus produced by many layers being applied onto one another to form a stack of slices. Advantageously the scattered radiation masks are then produced by an inventive system for additive manufacturing of a scattered radiation mask being toughened up and employed so that thin layers of minimum thickness are applied. To do this, the scattered radiation mask produced is built up for example on a vertically movable plate, which is lowered in the course of the production process and/or moved horizontally. Such an arrangement is known from the area of the additive manufacturing method, for example from 3D printing, in particular as a “core xy” arrangement. Basically it is conceivable, but not part of the present invention, to set the direction of production as perpendicular to the upper side.

A scattered radiation mask, regularly also referred to as a scattered radiation grid or a grid, serves to reduce undesired scattered radiation during x-ray imaging. The scattered x-ray radiation arises when x-ray radiation interacts with material and is scattered in various directions. This scattered radiation can greatly adversely affect the image quality in the x-ray diagnostics, in that it reduces the contrast and smudges details. The scattered radiation mask can in particular be embodied in the form of a grid or in the form of a strip.

The scattered radiation mask reduces the incident scattered x-ray radiation, in particular by the scattered x-ray radiation that does not strike the scattered radiation mask at right angles, interacting in particular with the x-ray radiation-absorbing second slice and/or with further x-ray radiation-absorbing slices and, when this occurs, the scattered x-ray radiation is at least attenuated, advantageously absorbed, as completely as possible. In other words the number of the x-ray photons of the scattered x-ray radiation after the scattered radiation mask is significantly reduced by comparison with the number of x-ray photons of the scattered x-ray radiation before the scattered radiation mask, is advantageously maximally minimized.

By comparison with this, the primary x-ray radiation in particular, i.e. x-ray radiation not scattered in material, but most highly attenuated, ideally passes through the x-ray radiation-transparent first slice or third slice, in particular without absorbent interaction with the second slice. In other words the number of x-ray photons of the primary x-ray radiation beyond the scattered radiation mask compared to the number of x-ray photons of scattered x-ray radiation in front of the scattered radiation mask is advantageously only slightly smaller due to the unavoidable physical interactions with the material of the second slice, preferably is essentially the same.

Whether an x-ray photon is absorbed by the scattered radiation mask as scattered x-ray radiation or is transmitted as primary x-ray radiation depends in particular on the angle of incidence of the trajectory on which the x-ray photon is located in relation to the surface of the scattered radiation mask. With a strip-shaped scattered radiation mask, the direction of the x-ray photons, for example parallel to the layers or perpendicular to the layers, typically additionally plays a role. The surface of the scattered radiation mask is in particular on the upper side of the scattered radiation mask.

The stack of slices forms the basic structure of the scattered radiation mask. A stack of slices refers to an arrangement of a number of layers lying above one another with different characteristics with regard to the x-ray radiation, in particular the x-ray radiation attenuation characteristic. The x-ray radiation attenuation characteristic defines the absorption rate of x-ray radiation. The stack of slices is in particular a stack of layers with a number of layers with different characteristics. A slice in particular has neighboring layers with the same x-ray radiation attenuation characteristics. Neighboring layers are in particular those layers that are applied directly to one another. Neighboring layers with the same characteristics in particular do not surround one or more layers with a different characteristic. The at least one layer of the stack of the first as well as the third slice and the at least one layer of the second slice differ in particular in their x-ray radiation attenuation characteristic. The at least one layer of the first and also the third slice in particular have a comparatively low x-ray radiation attenuation characteristic and thus a high x-ray radiation transmission characteristic. The at least one layer of the second slice in particular has a comparatively high x-ray radiation attenuation characteristic and thus a low x-ray radiation transmission characteristic.

The x-ray radiation-transparent slices each comprise one or more neighboring x-ray radiation-transparent layers and make possible a high transmission of the primary x-ray radiation. The x-ray radiation-absorbing slice comprises at least one or more neighboring x-ray radiation-absorbing layers and absorbs the scattered radiation as effectively as possible.

An x-ray radiation-transparent layer can consist of materials with a low atomic number, for example of plastics such as polyethylene, epoxy resin or polypropylene. These materials have a low x-ray radiation attenuation characteristic and thus let a majority of the primary x-ray radiation pass through them. As an alternative or in addition, materials with a low density such as foams and/or aerogels can be used. The use of a carrier matrix made of a plastic, preferably polyethylene, in particular represents a good option, which is also used frequently in additive manufacturing. This material polyethylene is moreover suitable as an x-ray radiation-transparent material without a proportion of an x-ray radiation-absorbing material.

An x-ray radiation-absorbing layer typically consists of materials with a high atomic number and high density. Examples of this are metals such as in particular lead or preferably tungsten, tantalum, rhenium, osmium, iridium, bismuth, platinum, thallium, mercury or gold. These materials absorb x-ray radiation effectively and can thus reduce the scattered x-ray radiation.

A planar layer refers to a thin, extended surface of the respective material. The thickness of such a layer can lie in the range of a few micrometers to a few hundred micrometers, while the lateral extent is far greater and typically lies in the range of centimeters or more. The thickness of the layer thus describes a strength of the layer. A layer can be understood in this context as a single, contiguous surface of the respective material, which is applied in a single production step. The thickness of such a layer can vary, depending on the material used and the desired characteristics, between the layers and/or within the layer. For example the thickness of the x-ray radiation-absorbing layer can lie in the range of 20 to 200 μm, while the thicknesses of the x-ray radiation-transparent layers can lie in the range of 50 to 500 μm, preferably 20 to 200 μm. The thickness of a layer can vary, depending on the material used and the desired characteristics.

The additive manufacturing method makes possible the precise production of complex structures by layered application of material, that is layer by layer. The additive manufacturing method is carried out in particular by the so-called “core xy” arrangement as part of the inventive system. In the manufacturing of the scattered radiation mask the individual layers are built up one after the other, wherein the layers for the x-ray radiation-transparent first and third slice as well as the x-ray radiation-absorbing second slice are applied one after another in the stack direction. In particular the at least one layer of the second slice is applied to the topmost or last layer of the layers of the first slice already produced and thereafter the at least one layer of the third slice is applied to the topmost or last of the layers of the second slice already produced. The order of production can also be reversed.

The additive manufacturing method can be a slice-by-slice construction method, in which the individual layers are applied one after the other. This method can make possible a precise control via the layer thicknesses and geometries. The layers can be applied using various techniques. One possibility can be the selective application of material by an application unit, for example with an application nozzle. Here the material can be applied in liquid or paste or powder form, in particular solid, or can subsequently be hardened. Another option can be the selective hardening of a powder bed by introduction of energy, for example by a laser, for example by selective laser sintering, or of another energy source. As a further method the powder applied can also be hardened by a chemical process, in which additional material is applied, for example by application, in particular spraying on, of a liquid, also called binder jetting. For the application of a layer it is conceivable for one or both of the material compositions to be applied as a liquid. In particular, X-ray radiation-transparent plastics, such as for example polyethylene, typically have a relatively low melting point of less than 300° C., while the x-ray radiation-absorbing materials, such as for example tungsten, can have comparatively high melt temperatures in the range of well over 1000° C., for example 3422° C. for tungsten.

The x-ray radiation-transparent first slice can have one or more x-ray radiation-transparent layers. The x-ray radiation-transparent third slice can have one or more x-ray radiation-transparent layers. The x-ray radiation-absorbing second slice can have one or more x-ray radiation-absorbing layers. It is conceivable for the number per slice to vary between the slices. The number per slice can in particular amount to 1, 2 or M>2. Preferably the neighboring layers of the stack of slices, i.e. the respective layers of various slices adjoining one another and the respective layers within a slice adjoining one another, are applied to one another in a planar manner in the stack direction via the additive manufacturing method.

It is preferred that an x-ray radiation-absorbing slice exclusively comprises x-ray radiation-absorbing layers and that an x-ray radiation-transparent slice exclusively comprises x-ray radiation-transparent layers. It is conceivable, in particular in respect of the choice of the manufacturing method for the one or more layers between an x-ray radiation-absorbing slice and an x-ray radiation-transparent slice, to have a medium x-ray radiation attenuation characteristic, for example on account of an unwanted contamination and/or for gradual setting of the x-ray radiation attenuation characteristic. The amount of the medium x-ray radiation attenuation characteristic lies in particular between the low x-ray radiation attenuation characteristic of the x-ray radiation-transparent slice and the high x-ray radiation attenuation characteristic of the x-ray radiation-absorbing slice.

Overall, the scattered radiation mask can have a plurality of slices with different x-ray radiation attenuation characteristics. Typically slices with a low x-ray radiation attenuation characteristic and slices with a high x-ray radiation attenuation characteristic alternate, in particular regularly, preferably periodically, in the direction of alternation. The direction of alternation of slices refers in particular to the direction in which the x-ray radiation-transparent slices and the x-ray radiation-absorbing slice alternate. The direction of alternation of the slices runs in particular in the direction in which materials, in particular slices, with a different x-ray radiation attenuation characteristic alternate.

The stack of slices, in addition to the first, second and third slice, can in particular have a plurality of slices. For example a further x-ray radiation-absorbing layer adjoins the x-ray radiation-transparent first slice, which is across from the x-ray radiation-absorbing second slice. As an alternative or in addition, another x-ray radiation-absorbing slice adjoins the x-ray radiation-transparent third slice, which is across from the x-ray radiation-absorbing second slice.

The stack direction of the layers relates in particular to the stack direction of the manufacturing method, i.e. to the direction in which the layers are applied in a planar manner above one another. The stack direction is typically perpendicular to the plane of the planar layer. In accordance with one or more example embodiments of the present invention, the stack direction runs essentially parallel to the upper side or surface of the scattered radiation mask. The stack direction thus in particular does not run perpendicular to the upper side or surface of the scattered radiation mask. In accordance with one or more example embodiments of the present invention, the direction of alternation of the slices corresponds to the stack direction of the layers.

An advantage of one or more example embodiments of the present invention thus lies in the fact that the critically thin structures are given by the layer thickness. It is especially advantageous that the neighboring layers of the stack of slices are or will be applied in stack direction as well as simultaneously in the direction of alternation, since the surface of the individual layers can be created with a very broad application of material, for example greater than 100 μm or in the range of 0.1 mm to 1 mm, but especially thin in the thickness direction. This advantageously enables extremely thin, in particular x-ray radiation-absorbing, layers and thus x-ray radiation-absorbing slices to be produced. As an alternative or in addition, the production time of the individual layers is reduced compared to conventional practice of building up the layers perpendicular to the upper side of the scattered radiation mask and thus perpendicular to the direction of alternation. This is because, for an x-ray radiation-absorbing slice, it has conventionally become necessary in particular in usual 3D printing methods to divide this slice into 5 or more individual spatial points per layer, which makes production of the scattered radiation mask with dimensions of for example 400 mm×400 mm in the prior art extremely expensive.

The scattered radiation mask described can thus offer a number of technical advantages overall. The use of the additive manufacturing method enables a very precise control of the layer thicknesses and geometries to be achieved. This can lead to an improved efficiency in scattered radiation reduction. Furthermore, the use of alternative materials instead of lead can improve the environmental compatibility of the scattered radiation mask. The option of producing complex structures can moreover make possible an optimization of the scattered radiation reduction for specific applications in x-ray imaging.

One form of embodiment makes provision for a minimum structure size within a layer to be larger than a minimum thickness of this layer. The minimum thickness of a layer can represent the smallest extent in the stack direction that can be achieved by production techniques for an individual layer. An example of this configuration can be a layer for which the minimum structure size within the layer amounts to 100 μm, while the minimum thickness of the layer can be 20 μm or 50 μm. Essentially an anisotropic additive manufacturing method described in this form of embodiment, in which the layers are very thin, for example a layer thickness is in the order of magnitude 20 μm, while the material application in the two directions orthogonal to the stack direction has far rougher minimum structure sizes of greater than 20 μm, for example in the range of 50 μm to 1000 μm. The minimum structure size can relate to the smallest lateral dimension that can be manufactured within a layer of the scattered radiation mask. This size can be determined by the resolution of the additive method manufacturing used. For example, with a 3D printing method, the minimum structure size can be influenced by the diameter of the cross section of the application nozzle of the application unit of the system for additive manufacturing or by the precision of the positioning of the application unit. The minimum structure size can, as an alternative or in addition, depend on factors such as the viscosity of the material used, the surface tension and/or the hardening characteristics. For example in a stereolithography method the slice thickness can be controlled precisely by the penetration depth of the hardening light, while the lateral resolution can be restricted by the focus diameter of the laser. As an alternative, in the case of a powder bed fusion method, the slice thickness can be determined by the height of the powder covering applied, while the minimum structure size can depend on the grain size of the powder and the precision of the energy input.

The relationship between minimum structure size and minimum thickness can have various effects on the performance of the scattered radiation mask. A larger minimum structure size compared to the minimum thickness can lead to an improved mechanical stability of the layer. This can be especially important for the x-ray radiation-absorbing slices, since these consist of thicker materials and can therefore be more susceptible to structural weaknesses. Moreover these forms of embodiment can make possible a layer thickness or slice thickness that is as small as possible. This can be of advantage for the optimization of the absorption characteristics of the scattered radiation mask.

This configuration can make possible an optimization of the scattered radiation mask for specific applications in x-ray imaging.

One form of embodiment makes provision for a thickness of the second slice to be less than 200 μm, in particular less than 100 μm, preferably less than 50 μm, especially advantageously less than 25 μm. The thickness of the x-ray radiation-absorbing second slice is an important parameter for the performance of the scattered radiation mask. Preferably a thinner slice can minimize the absorption of the primary x-ray radiation, while at the same time an effective reduction of the scattered radiation is achieved. Examples of specific thicknesses within the said area can be 160 μm, 100 μm, 50 μm, 40 μm, 25 μm or 20 μm. A thickness of 100 μm can for example represent a good compromise between absorption capability and overall thickness of the scattered radiation mask. A thickness of 100 μm can make possible an especially low absorption of the primary x-ray radiation, while a thickness of 20 μm can lead to an especially efficient scattered radiation mask. The choice of the specific thickness can depend on various factors, such as for example the absorption material used, the desired scattered radiation reduction and the specific requirements of the x-ray imaging application. A thinner x-ray radiation-absorbing slice can also increase the flexibility of the scattered radiation mask, which can be advantageous in particular applications. The thickness of the second slice cannot be less than the thickness of the at least one layer of the second slice. The thickness of the second slice typically amounts to at least the sum of the thickness of the layers of the second slice.

One form of embodiment makes provision for the second slice to have a maximum of one layer and/or wherein the first slice and optionally the third slice can each have a maximum of one layer. In this case the thickness of the one layer in particular amounts to less than 200 μm, in particular less than 100 μm. This form of embodiment describes inter alia a scattered radiation mask with a simplified slice structure, in which each of slices-the x-ray radiation-transparent first slice, the x-ray radiation-absorbing second slice and the x-ray radiation-transparent third slice - consists of a maximum of one layer. The use of a maximum of one layer per slice can simplify the manufacturing process of the scattered radiation mask. In an additive manufacturing method this can mean that each slice is applied in a single pass, which can reduce the production time and the material consumption. Moreover this procedure can increase the precision of the slice thicknesses, since inaccuracies that can arise through the multiple application of layers are avoided. Since each slice consists of a single layer, the composition and structure of the material within the slice can be more homogenous, which can lead to a more even absorption or transmission of the x-ray radiation. One possible arrangement of the layers can appear as follows: a single layer of the x-ray radiation-transparent first slice with a thickness of 100 μm, followed by an individual layer of the x-ray radiation-absorbing second slice with a thickness of 20 μm, and finally an individual layer of the x-ray radiation-transparent third slice with a thickness of 100 μm. By variation of the thickness and composition of the individual layers, the mask can be optimized for specific applications in x-ray imaging without increasing the complexity of the manufacturing process.

One form of embodiment makes provision for a cross section of a layer to be embodied in the shape of a trapezoid in the width direction. A trapezoidal cross section can be understood in this connection as a geometrical shape in which the upper and lower edge of the layer can run parallel to one another as well as parallel to the stack direction, but have different lengths. The side edges of the layer can run at an angle in this case and connect the upper edge with the lower edge. The width direction is perpendicular to the stack direction and also perpendicular to the upper side of the scattered radiation mask. The trapezoidal shape is in particular a truncated wedge shape. It is conceivable for a cross section of a number of layers or of all layers to be embodied in a trapezoidal shape in the width direction. The cross section of two or more layers can be different, in particular have at least have one different internal angle, that is to not cover the same area. For example the in particular trapezoidal cross sections can be aligned differently, in particular slanted. The variation in thickness can be designed differently for various layers of the stack of slices. For example in an x-ray radiation-transparent layer a different thickness variation can be used from that used in an x-ray radiation-absorbing layer. This can make possible a precise tailoring of the absorption characteristics of the scattered radiation mask.

The trapezoidal embodiment of the cross section of a layer can also be achieved by various production methods. In additive manufacturing methods for example the amount of the material applied during the application of a layer can be explicitly varied in order to create the desired trapezoidal shape. In particular a thickness of a layer of the stack of slices can be varied via the additive manufacturing method in the width direction. In a method with material applied as a liquid or a paste for example, the amount of the material applied can be explicitly changed in the width direction of the layer. This can be carried out by adapting the flow of material, the speed of movement of the application unit or by a combination of the two parameters. In a powder-based additive manufacturing method, the variation in thickness can be achieved by selective hardening of different amounts of powder material in various areas of the layer. This can be realized by variation of the amount of energy that is used for hardening the powder, or by traveling over specific areas multiple times.

The inclination of the side edges of the trapeze can vary and be adapted to the specific requirements of the scattered radiation mask. For example an angle of inclination of between 0.1° and 45° can be chosen. A trapezoidal cross section of a layer can offer various potential advantages for the performance of the scattered radiation mask. On the one hand this form can contribute to a focusing of the scattered radiation mask. The angled side edges can serve as a type of guide for the x-ray radiation and reduce undesired scattered x-ray radiation. It is significantly advantageous for the scattered radiation mask to be focused in such a way that in particular the x-ray radiation-absorbing slices of the scattered radiation mask are aligned to the x-ray radiation source. This means in particular that slices in the center of the scattered radiation mask are at right angles to the surface, while slices with an increasing distance to the center are inclined ever more in the direction of the x-ray radiation source. This geometrical relationship is shown in FIG. 3. This can lead to an enhanced image quality in the x-ray imaging. The trapezoidal embodiment of the cross sections can contribute to the optimization of the absorption characteristics of the scattered radiation mask. The variation of the thickness within an in particular x-ray radiation-absorbing layer enables a gradual change in the absorption characteristics to be achieved. This can be especially useful for adapting the mask to specific x-ray spectra or imaging modalities. As an alternative or in addition the trapezoidal form of a layer can contribute to reducing reflections and scatterings at the boundary surfaces between the layers. The use of a layer with trapezoidal cross section can in particular increase the flexibility in the design of the scattered radiation mask. Variation of the trapeze shape in various layers enables complex three-dimensional structures, which are adapted to the specific requirements of the x-ray imaging, to be created within the mask.

One form of embodiment makes provision for the x-ray radiation-transparent slices to be embodied from a first material composition and the x-ray radiation-absorbing slice from a second material composition, wherein the first material composition and the second material composition are embodied differently from one another with regard to a proportion of a material. The first material composition and the second material composition can in particular be embodied differently from one another with regard to a proportion of exclusively one material or a number of materials. A proportion can lie between 0% and 100%. Proportions differ by definition from one another in this form of embodiment, when, on account of the difference the x-ray radiation attenuation characteristic of the first material composition differs from the x-ray radiation attenuation characteristic of the second material composition in such a way that the first material composition is x-ray radiation-transparent and that the second material composition is x-ray radiation-absorbent. A material composition comprises a single material or a specific combination of materials, which together determine the characteristics of a slice. In particular the first material composition can comprise just one material, while the second material composition can comprise two or more materials. As soon as a material composition does not comprise a material of the other material composition, the two material compositions differ, since the proportion of the one material is equal to zero in the one material composition and not equal to zero in the other material composition. The material composition can comprise various materials, for example elements, compounds or structures in different proportions. The first material composition of the x-ray radiation-transparent slices can for example consist of lightweight materials with a low atomic number. Possible materials for this can be plastics such as polymers, polyethylene, epoxy resin, polypropylene or acrylic glass. In addition these materials can be provided with additives or fillers in order to achieve specific characteristics. The second material composition of the x-ray radiation-absorbing slice can comprise materials with a high atomic number. Such materials can be metals such as lead, tantalum, tungsten, rhenium, osmium, iridium, bismuth, platinum, thallium, mercury or gold, which can be embedded in the form of fine powders or nanoparticles into a carrier matrix, for example made of plastic.

One form of embodiment makes provision for the first material composition to have hollow glass spheres and/or an aerogel. Hollow glass spheres, also known as micro hollow glass spheres or “micro balloons” or glass microspheres, are microscopically small hollow spheres of glass. These spheres typically have a diameter of a few micrometers, for example 80 μm, up to a few hundred micrometers and consist in particular of a thin glass shell, which encloses a gas volume or vacuum. Because of their structure, hollow glass spheres have a very low density, typically in the range of 0.125 to 0.6 g/cm³. This low density, in combination with the characteristics of the glass, makes hollow glass spheres an outstanding material for x-ray radiation-transparent slices. Technically, by mixing the hollow glass spheres with an epoxy resin or with a liquid, for example thermoplastic, a plastic material with very low density is able to be manufactured. With high concentrations of hollow glass spheres a type of foam with a density of below that of water (1.0 g/cm3) is produced. Advantageously the hollow glass spheres are employed for scattered radiation mask production for reduction of density or absorption at a number of points: The paper is completely replaced by the hollow glass spheres, for example the hollow glass spheres are embedded in a thermoplastic plastic or an epoxy resin in accordance with this form of embodiment. As an alternative or in addition adhesive materials for cover coatings and/or the material of the cover coatings of the scattered radiation masks can be filled with hollow glass spheres. Overall the use of the hollow glass spheres makes possible a very marked reduction in undesired absorption of the primary x-ray radiation in the x-ray radiation-transparent slices. In hollow glass spheres embedded in plastics in particular, the very low density able to be achieved makes possible a marked reduction of absorption of primary x-ray radiation. In particular the replacement of the problematic natural product paper by a plastic filled with hollow glass spheres allows scattered radiation masks to be produced significantly more effectively and reliably.

For the manufacture of layers with hollow glass spheres a method can be used in which the hollow glass spheres are embedded into a carrier matrix, in particular consisting of a plastic. This can be done by mixing the hollow glass spheres with the plastic, in particular a liquid plastic, and by subsequent hardening of the plastic. As an alternative the hollow glass spheres can also be introduced into a powder bed and subsequently bound by sintering or melting.

As an alternative or in addition, hollow glass sphere-filled materials can also be supplemented or replaced by aerogels with even lower density. Aerogels are highly porous solids with a low density. They consist for a large part of air (often over 95% of the volume) and have a network-like structure of nanoparticles connected to one another. Aerogels can be manufactured from various materials, wherein silica aerogels are the most widely known. The low density and the open structure likewise make aerogels an excellent material for x-ray radiation-transparent slices. These aerogels are typically available in far greater thicknesses of a few mm. It is conceivable to employ aerogels in additive manufacturing, in particular in 3D printing. Aerogel layers in the range of 100 μm thickness in particular represent an addition or alternative to the use of hollow glass spheres.

In the use of aerogels a sol-gel method can be applied, in which first of all a gel is manufactured and the solvent is subsequently removed by overcritical drying, without destroying the pore structure. An alternate method is freeze drying, in which the solvent is removed by sublimation.

In both cases the additive manufacturing method can be adapted in order to make optimum use of the special characteristics of these materials.

The use of hollow glass spheres and/or aerogels in the first material composition can significantly improve the x-ray transparency of the corresponding slices, i.e. reduce the x-ray radiation attenuation characteristic to the benefit of a higher x-ray radiation transmission characteristic, which in turn can increase the efficiency of the scattered radiation mask. The reason for this is that both materials have a high proportion of air or gas, which lets x-ray radiation pass through almost unhindered. At the same time these materials can positively influence the mechanical stability and other important characteristics of the slices. In particular the lower density of these materials can contribute to a reduction in the overall weight of the scattered radiation mask, which can be advantageous in specific applications. For example, special application nozzles can be used, which make possible an even distribution of the hollow glass spheres or a controlled deposition of the aerogel.

One form of embodiment makes provision for the second material composition to have lead, tantalum, tungsten, rhenium, osmium, iridium, bismuth, platinum, thallium, mercury or gold. Especially advantageously these elements, i.e. the said metals of the 6th period, are embedded in the carrier matrix described and employed in the inventive system for additive production. Through this, almost complete freedom of geometrical design of scattered radiation masks preferably exists. The second material composition in particular forms the x-ray radiation-absorbing slice of the scattered radiation mask. The use of the aforementioned materials with a high atomic number and high density is decisive for an effective absorption of x-ray radiation. The said elements have these characteristics and can therefore be especially suitable for use in the x-ray radiation-absorbing slice.

Because of its high density of around 11.3 g/cm³ and its good absorption characteristics, lead can be used for x-ray radiation. Lead, as an element of the 6th period in the periodic system, has the highest relevant atomic number for x-ray radiation absorption. Because of their increasingly low atomic numbers the following stable elements of the 6th period come into question in particular as a substitute for lead, under certain conditions also thallium and mercury, despite their toxicity. In particular the preferred elements tantalum, tungsten, rhenium, osmium, iridium, bismuth, platinum or gold elements come into question as a substitute for lead. As well as the atomic number of the preferred elements, their density is very relevant. With the given material thickness for lead (for example 20 μm) the material thickness needed for the preferred elements is able to be calculated. The results of the calculations are illustrated in FIG. 11. Tantalum has a higher density of around 16.7 g/cm3 than lead. Tungsten, with a density of around 19.3 g/cm3 , can offer an outstanding absorption capability for x-ray radiation. Rhenium, with a density of around 21.0 g/cm3 , can offer a very high absorption capability. Osmium has the highest density of around 22.6 g/cm3 of the said elements. Iridium, with a density of around 22.6 g/cm3 , can likewise have a very high absorption capability. Platinum, with a density of around 21.5 g/cm3 can offer an excellent absorption capability for x-ray radiation. Gold, with a density of around 19.3 g/cm3 can offer a very good absorption capability. Mercury, with 13.6 g/cm3 and thallium with 11.7 g/cm3 , has a higher density than lead, while the density of bismuth, with 9.8 g/cm3 , is lower.

The result is that all the preferred elements mentioned above can be employed more advantageously than lead, since, with a simultaneous screening effect, the result is smaller material thicknesses. Of the said elements, tungsten is the one which above all comes into consideration because of its relatively low price. Specifically the elements osmium, gold and platinum, because of their comparatively high prices, can be excluded for commercial reasons for use in scattered radiation masks.

These materials can be embedded into the scattered radiation mask in various ways. One option can be the embedding of fine powders or nanoparticles of these materials into a carrier matrix or alloys with such materials.

The use of these materials in the second material composition can offer various advantages. On the one hand a high absorption efficiency can be achieved, which can lead to an effective reduction of the scattered radiation. On the other hand, through the choice of the material and the form of processing, the thickness of the absorbing slice can be optimized, which can lead to a reduction in the overall thickness of the scattered radiation mask. What is more, the use of alternative materials to lead can enhance the environmental compatibility of the scattered radiation mask.

With known methods many metals can be deposited chemically or electrochemically from an aqueous solution. In the actual choice of the element it is important that the element is able to be deposited industrially. Here in particular, despite its high price, gold is a good alternative, since corresponding methods of depositing gold are well established in the electrical engineering industry for refinement of contact surfaces. Only elements of the 6th group from rhenium to bismuth can be deposited electrochemically from an aqueous solution. Because of its chemical makeup, tungsten cannot be deposited from an aqueous solution. Rhenium comes into question in accordance with one or more example embodiments of the present invention for an electrolytic deposition and also has the lowest price of the metals mentioned above. However, with methods that have recently become known (see Dominik Höhlich et. al.: Simultaneous Electrodeposition of Silver and Tungsten from [EMIm]Cl:AlCl3 Ionic Liquids outside the Glove Box; Coatings 2020, 10(6), 553; https://doi.org/10.3390/coatings10060553, DOI: 10.7395/2020/Hoehlich and the patent DE 10 2014 118 593 A1), tungsten can be deposited together with nickel or silver from an ionic (non-aqueous) solution. In particular a corresponding deposition of tungsten or tungsten in conjunction with another metal is possible.

Tungsten comes into question as a relatively low-cost element. Since tantalum is also non-toxic as a material and it still has a slightly better x-ray radiation attenuation characteristic than lead, it can also be provided as a material for the x-ray radiation-absorbing slices. In particular tungsten-rhenium alloys can be employed. The advantage of these alloys is that the mixture ratio of the two elements typically has no role to play in the x-ray radiation attenuation characteristic, since this is almost identical for both elements. Alloys consisting of tungsten with for example copper, nickel and/or iron that can be easily shaped also represent a further option. Since these alloys are technically offered with a typical tungsten content of 90% to 95%, the low x-ray radiation attenuation characteristic of the added light elements, copper for example has around 9% of the absorption capability by volume of lead, typically only leads to a slight increase in the thickness of x-ray radiation-absorbing layers needed.

As an alternative to the alloys with the preferred elements, mixtures of powders of the preferred elements with other binder substances can be used for manufacturing the x-ray radiation-absorbing slices. As binders or matrix material, both metals, such as in particular silver or tin come into question, and also plastics. The embedding of tungsten powder into tin is known from DE 60 2004 000 309 T2. As an alternative, plastics in particular are suitable as a material for embedding of tungsten powder. Commercial materials with a tungsten powder content of over 90% in a carrier matrix made of polyethylene are available. These materials have densities of up to 15 g/cm3.

One form of embodiment makes provision for the first material composition to have a plastic matrix and for the second material composition to have the same plastic matrix. The plastic matrix is in particular a carrier matrix made of plastic. The two material compositions have the same plastic matrix, which does not mean that the two material compositions are identical. The two material compositions can additionally each have a material that is not contained in the other respective material composition. It is conceivable for the first material composition to consist of the plastic matrix and for the second material composition to have a proportion greater than zero of an x-ray radiation-absorbing metal, such as for example the aforementioned materials, which is embedded into the same plastic matrix. The plastic matrix refers to a polymer material, which serves as a basic structure or binding mechanism or means for other materials. In the scattered radiation mask of this form of embodiment this carrier matrix will be used both in the x-ray radiation-transparent slice and also in the x-ray radiation-absorbing slice. For the x-ray radiation-transparent slices in particular, the plastic matrix can consist of a material with a low atomic number. These materials have a low absorption of x-ray radiation and can therefore make possible a high transmission of the primary x-ray radiation. The same plastic matrix is used in the x-ray radiation-absorbing slices in this form of embodiment, in particular with additions of materials with a high atomic number however. These additions can in particular be embedded into the matrix in the form of fine powders or nanoparticles. Preferably tungsten or tantalum powder can be worked into the plastic matrix in order to increase the x-ray absorption. The use of the same plastic matrix in both material compositions can offer a number of advantages. First on all an improved adhesion between the slices can be achieved, since the chemical compatibility between the materials is increased. This can lead to an improved structural integrity of the overall scattered radiation mask. Furthermore, the use of the same matrix can simplify the manufacturing of the scattered radiation mask. With additive production methods for example, the same basic material can be used for all slices, wherein only one addition or the additions is or are varied for the x-ray radiation-absorbing slice. This can design the production process more efficiently and avoid possible compatibility problems between various matrix materials. Moreover the use of a uniform matrix can lead to a more even thermal extent of the various slices. This can reduce thermal stresses and possible deformations of the scattered radiation mask with changes in temperature. The choice of the specific plastic matrix can depend on various factors, such as for example the desired mechanical stability, the workability in the additive production process and the compatibility with the additional materials used for the absorbing slice.

Ideally the machine has at least two application nozzles, with which different materials are applied: An x-ray radiation-absorbing material that is as strong as possible with one of the two application nozzles and a material that is as x-ray radiation-transparent as possible with the other of the two application nozzles. Basically it is conceivable for the two application nozzles to apply, one after another and alternating layer by layer, either x-ray radiation-absorbing material or x-ray radiation-transparent material.

The scattered radiation mask is produced from an alternating construction of layers of the two materials. As a heavily x-ray radiation-absorbing material preferably a meltable matrix, which is filled with a high proportion of fine tungsten powder or other powders of elements of the 6th period of the periodic system of elements, comes into question. In particular, x-ray radiation-absorbing means as x-ray radiation-proof as possible by comparison with x-ray radiation-transparent.

One form of embodiment makes provision for a layer of the first slice and/or the third slice in the width direction to have an x-ray radiation-absorbing strip. The at least one x-ray radiation-absorbing strip covers a maximum of up to 50%, for example less than 30% of the x-ray radiation-transparent layer. The x-ray radiation-absorbing strips can be integrated into the otherwise x-ray radiation-transparent slices. These strips can consist of the same material as the x-ray radiation-absorbing second layer or of another material with a higher x-ray absorption. The strips can be created for example by selective application of the absorbing material during the additive manufacturing process. The x-ray radiation-absorbing strips can extend over the entire width direction of the layer or only over a part of it. The purpose of these additional absorbing strips can consist of improving the scattered radiation reduction in a further dimension. While the x-ray radiation-absorbing second slice primarily reduces scattered radiation in one direction, the additional strips in the x-ray radiation-transparent layers can reduce scattered x-ray radiation in a direction orthogonal to this. The performance of the scattered radiation mask can be influenced in multiple respects by this configuration. On the one hand an improved scattered radiation reduction in two dimensions can be reached, which can lead to a higher image quality in the x-ray imaging. On the other hand, the flexibility in the design of the scattered radiation mask is increased, since the density and/or arrangement of the x-ray radiation-absorbing strips can be adapted to specific requirements. Through this the scattered radiation mask in particular takes the form of a grid. The extent in the width direction as well as the length direction and the spacing of the x-ray radiation-absorbing strips can be varied in order to obtain the desired absorption characteristics. For example, the strips can have an extent in the longitudinal direction of the layer of 50 to 500 μm and be arranged with a spacing of 1 to 10 mm. The thickness of the strip can correspond to the thickness of the layer into which it is integrated, or can differ therefrom. The x-ray radiation-absorbing strip can be integrated into the x-ray radiation-transparent layer using various methods. In additive production methods the absorbing material can be applied specifically at the desired positions. As an alternative prefabricated absorbing strips can be embedded into the transparent slices. This configuration can make possible an optimization of the scattered radiation mask for specific applications in x-ray imaging. By adaptation of the strip geometry and arrangement the mask can be adapted to various imaging modalities and examination objects. In summary, the form of embodiment described thus has the following advantages: By controlling the system for additive manufacturing, individual strips consisting of x-ray radiation-absorbing material can be integrated into the layers consisting of x-ray radiation-transparent material. Conditional on the process, these strips can also have a greater extent than the layer thickness in the longitudinal direction, in particular because of the anisotropically minimum structure size, scattered radiation masks can be produced in the form of grids. Since the layers and number of absorbing strips in the individual x-ray radiation-transparent layers can be predetermined as any given value, a scattered radiation mask, the geometry of which is optimally adapted to the planned possible uses, can be produced.

One form of embodiment makes provision for x-ray radiation-absorbing strips of neighboring layers to be combined to form an N-cornered structure, with N not equal to 4. An N-cornered structure refers in this context to a geometrical form with N corners and N sides, which is formed by the arrangement of the x-ray radiation-absorbing strips in neighboring layers. The term “N not equal to 4” means that the number of corners and sides does not amount to four, but can be another whole number greater than two. In particular the N-cornered structure is not a rectangle or square. Examples of such N-cornered structures can be triangles (N=3), pentagons (N=5), hexagons (N=6) or more complex polygons with a higher number of corners. A scattered radiation mask of this form of embodiment has a grid form. The specific choice of N can depend on the desired absorption characteristics and the intended application of the scattered radiation mask. The formation of these N-cornered structures can be achieved by a precise arrangement of the x-ray radiation-absorbing strips in consecutive layers. For example, in a first layer strips can be arranged at a specific position in relation to the longitudinal extent, while in the layer opposite it they can be arranged at an offset position. The overlaying of these strips enables the N-cornered structures to be produced.

This form of embodiment can influence the performance of the scattered radiation mask in multiple respects. On the one hand an improved scattered radiation reduction in various directions can be achieved, since the N-cornered structures can absorb scattered radiation from different angles. On the other hand this arrangement can lead to a more even distribution of the absorption over the surface of the scattered radiation mask, which can reduce potential artifacts in the x-ray imaging.

The manufacturing of such a scattered radiation mask with N-cornered structures can be realized by additive production methods. In these methods the x-ray radiation-absorbing strips can be precisely positioned in each layer in order to create the desired N-cornered structures. These methods can make possible a high precision in the creation of the N-cornered structures. The size of the N-cornered structures can be varied in order to adapt the absorption characteristics of the scattered radiation mask to specific requirements. Smaller structures can make possible a finer control of the scattered radiation reduction, while larger structures are possibly easier to produce and overall bring about less x-ray radiation absorption.

An inventive arrangement for x-ray imaging has
an inventive scattered radiation mask,
an x-ray radiation source,
an x-ray detector and
an examination area between the x-ray radiation source and the x-ray detector,
wherein the scattered radiation mask is arranged between the examination area and the x-ray detector and is aligned in such a way that the stack direction of the stack of slices is parallel to the x-ray detector.

An x-ray radiation source can be an apparatus that creates x-ray radiation. The x-ray radiation source can be embodied to create x-ray radiation with a specific energy or a specific energy spectrum. The x-ray radiation source can in particular be an x-ray generator. The x-ray generator in particular comprises an evacuated housing, in which in particular an anode and a cathode are arranged. The cathode in particular comprises an electron emitter, by which free electrons can be accelerated in the direction of the anode. When the accelerated electrons strike the anode in particular the x-ray radiation is generated. The electron emitter can in particular be a cold emitter, for example a field effect emitter, or a thermionic, for example a helical or sheet metal emitter. An x-ray detector can be an apparatus that detects the x-ray radiation and converts it into an electrical signal. The x-ray detector can for example be a flat-panel detector, a row detector or an individual pixel detector. The x-ray detector can consist of various materials such as scintillators in combination with photodetectors or direct-converting semiconductor materials. An examination area can be a space or volume between the x-ray radiation source and the x-ray detector, in which an object to be examined can be placed. The object to be examined can for example by a human body, an animal, a technical component or any other given object. For x-ray imaging a patient is typically located in the examination area. The arrangement of the components can be designed so that the x-ray radiation source emits x-ray radiation, which passes through the examination area to strike the x-ray detector. The scattered radiation mask can be positioned between the examination area and the x-ray detector in order to reduce scattered radiation that arises in the examination area.

Since the inventive arrangement has the inventive scattered radiation mask, it shares the advantages previously described. The alignment of the scattered radiation mask with the stack direction of the stack of slices parallel to the x-ray detector can serve to optimize the efficiency of the scattered radiation reduction. The described arrangement for x-ray imaging can make possible an enhanced image quality, in that the scattered radiation is effectively reduced. The precise positioning and alignment of the scattered radiation mask in relation to the x-ray detector and the examination area can lead to an optimized scattered radiation reduction with simultaneous minimization of the absorption of the primary x-ray radiation.

The inventive arrangement can in particular be suitable for medical or technical x-ray imaging. Applications of medical x-ray imaging are in particular angiography, computed tomography, mammography and/or fluorescent radiography. Applications of technical x-ray imaging can in particular be materials testing, safety checks and/or customs checks.

An x-ray imaging system can comprise the inventive arrangement as well as for example at least one control computer, which can control the x-ray radiation source for the x-ray imaging. The x-ray imaging system can in particular be embodied for medical and/or technical x-ray imaging.

An inventive system for additive manufacturing of a scattered radiation mask for reduction of scattered x-ray radiation, in particular for x-ray imaging, has an application unit, which is embodied to carry out the inventive manufacturing method.

An application unit can be an apparatus which is embodied to apply material in layers in order to create a three-dimensional structure. The application unit can comprise various components, such as for example one or more application nozzles, extruders or other mechanisms for precise application of material. The application unit can be embodied to process and to apply various materials. These materials can comprise x-ray radiation-transparent materials of the first material composition for the first and third slice as well as x-ray radiation-absorbing materials of the second material composition for the second slice. The application unit can have mechanisms available to switch between various materials or to apply a number of materials at the same time. The system can be configured so that the application unit applies layers in a planar manner onto one another in the stack direction to form a stack of slices. In such cases the system can ensure that the stack of slices has an x-ray radiation-transparent first slice, an x-ray radiation-absorbing second slice and an x-ray radiation-transparent third slice, wherein each of these slices has at least one planar layer. The system can comprise control mechanisms, which make it possible to precisely control the thickness of the layers. This can be especially important for the manufacturing of scattered radiation masks with very thin absorbing slices. The system can have positioning systems available, which make possible a precise movement of the application unit in three dimensions. This can make possible an exact control over the geometry and structure of the scattered radiation mask manufactured. The system can also comprise systems for monitoring and quality control of the manufacturing process. These can for example include sensors for measuring the slice thickness or for checking the material composition. Such a system for additive manufacturing of a scattered radiation mask can make possible a high flexibility in manufacturing. The geometry and composition of the scattered radiation mask can be easily adapted in order to fulfill various requirements in x-ray imaging. Moreover, the additive production can make possible the manufacturing of complex internal structures that would be difficult to realize with conventional production methods. In addition the system can be embodied to vary the thickness of a layer of the stack of slices in the width direction. This can be achieved by precise control of the application of material during the additive manufacturing process, for example by adapting the material flow and/or the speed of movement of the application unit.

One form of embodiment makes provision for the application unit to have at least one application nozzle with an oval or rectangular cross section. The cross section of the application nozzle relates to the opening of the application nozzle from which the material emerges. The cross sections of a number of application nozzles can differ in shape and/or size. An application nozzle with an oval or rectangular cross section can offer a number of advantages for the manufacturing process and for the resulting structure of the scattered radiation mask. By contrast with a conventional round nozzle, an oval or rectangular nozzle can apply a wider material track in one single pass. This can reduce the production time and increase the efficiency of the manufacturing process.

The oval or rectangular cross section of the application nozzle can make it possible to apply material slices with a controlled width and thickness. The form of the nozzle can be chosen so that the material track applied corresponds to the desired geometry of the scattered radiation mask. For example a rectangular nozzle with a side ratio of 10:1 can apply a material track that is 10 times as wide as it is thick.

The use of an oval or rectangular application nozzle can also contribute to improving the surface quality of the scattered radiation mask manufactured. The wider application of material enables surface unevenness that can arise from the layer-by-layer application, to be reduced. This can lead to a smoother surface of the scattered radiation mask, which can be advantageous for the performance in x-ray imaging. Moreover, the form of the application nozzle can be adapted to the specific requirements of the various layers of the scattered radiation mask. For the x-ray radiation-transparent layers for example, a wider application nozzle can be used in order to make possible a rapid coverage of large surfaces. For the x-ray radiation-absorbing layers a narrower nozzle can be employed in order to guarantee a more precise control of the slice thickness.

The oval or rectangular shape of the application nozzle can also contribute to optimizing the internal structure of the scattered radiation mask. By explicitly aligning the application nozzle during the application, material chains can be created that are oriented in a specific direction. This can be used in order to control the absorption characteristics of the mask in various directions. A system for additive manufacturing of a scattered radiation mask with an application unit, which has at least one application nozzle with an oval or rectangular cross section, can make possible an improved control of the manufacturing process and the resulting mask structure. This can contribute to an optimization of the performance of the scattered radiation mask in the x-ray imaging, in particular through the focusing of the scattered radiation mask.

One form of embodiment makes provision for the system to a have a tilt apparatus and for the application unit and also for the tilt apparatus to be arranged in such a way that a tilt angle of the application unit is able to be changed relative to a layer already applied by the tilt apparatus. The tilt apparatus can be a mechanical system that makes it possible to incline the application unit about one or more axes. This tilt apparatus can for example consist of joints, hinges or pivot apparatuses, which allow a precise control of the angle of the application unit. The arrangement of the application unit and the tilt apparatus can be designed so that a change in the tilt angle of the application unit relative to a layer that has already been applied is possible. This means that the application unit can be positioned at various angles to the surface of the last layer of material applied. Through the tilt axis of the tilt apparatus the scattered radiation mask arising for example on a vertically movable plate can be tiltable by the tilt apparatus with different tilt angles relative to the surface in which the application unit is moving. As an alternative or in addition, the application unit can be tiltable against the horizontal by the tilt apparatus. The change in the tilt angle can be undertaken by a manual setting or by an automated control. An automated control can for example be realized by servo motors or actuator drives that are connected to a control unit. This control unit can adapt the tilt angle based on pre-programmed parameters or in real time during the production process. The variable tilt angle of the application unit can make it possible to manufacture focused scattered radiation masks. With a focused scattered radiation mask the absorbing structures are aligned so that they are aligned to a specific point, typically the x-ray source. This can be achieved by a gradual change in the angle of the applied layers.

The use of the tilt apparatus enables the angle of each layer applied to be controlled precisely. For example the tilt angle can be set so that the layers in the center of the mask are applied parallel to the surface, while the layers are increasingly tilted inwards toward the edge. This can lead to an improved efficiency of the scattered radiation mask, since the absorbing structures are aligned optimally to the x-ray source. Moreover, the tilt apparatus can contribute to improving the surface quality of the manufactured scattered radiation mask. Adaptation of the tilt angle enables the application of material to be controlled so that overhangs or undercuts are avoided, which can lead to a smoother surface and an improved structural integrity of the mask. The use of a tilt apparatus in the system for additive manufacturing of a scattered radiation mask can make possible an increased flexibility and precision in the manufacturing process. This can contribute to an improved performance of the scattered radiation mask in x-ray imaging, in that the geometry and alignment of the absorbing structures can be adapted optimally to the specific requirements of the respective application.

An inventive use of a system with an application unit for additive manufacturing of a scattered radiation mask for reduction of scattered x-ray radiation, in particular for x-ray imaging, is likewise part of the present invention. This use relates to the use of the system described above or of other systems with an application unit for additive manufacturing for the method for manufacturing the inventive scattered radiation mask. Each system with such an application unit can typically be used to build up the various slices of the scattered radiation mask precisely and efficiently.

Features, advantages or alternative forms of embodiment mentioned in the description of the apparatus are likewise to be transferred to the method and vice versa. In other words claims for the method can be further developed with features of the apparatus and vice versa. In particular the inventive apparatus can be used in the method.

### BRIEF DESCRIPTION OF THE DRAWINGS

Exemplary embodiments of the present invention are shown in the drawings and are described in greater detail below. The same reference numbers are used for the same features in different figures.

In the figures:

FIG. 1 shows a schematic diagram of an arrangement for x-ray imaging;

FIG. 2 shows a schematic diagram of an arrangement for x-ray imaging with an object in the examination area;

FIG. 3 shows a schematic diagram of an inventive arrangement for x-ray imaging with a scattered radiation mask;

FIG. 4 shows a perspective view of an inventive scattered radiation mask for reduction of scattered x-ray radiation;

FIG. 5 shows a perspective detailed view of an inventive scattered radiation mask for reduction of scattered x-ray radiation;

FIG. 6 shows a perspective detailed view of a first exemplary embodiment of the scattered radiation mask;

FIG. 7 shows a flow diagram of an inventive method;

FIG. 8 shows a perspective view of an inventive system for additive manufacturing of a scattered radiation mask;

FIG. 9 shows a schematic side view of a first exemplary embodiment of the system;

FIG. 10 shows a schematic side view of a second exemplary embodiment of the system and

FIG. 11 shows a table with materials for the x-ray radiation-absorbing layers.

### DETAILED DESCRIPTION

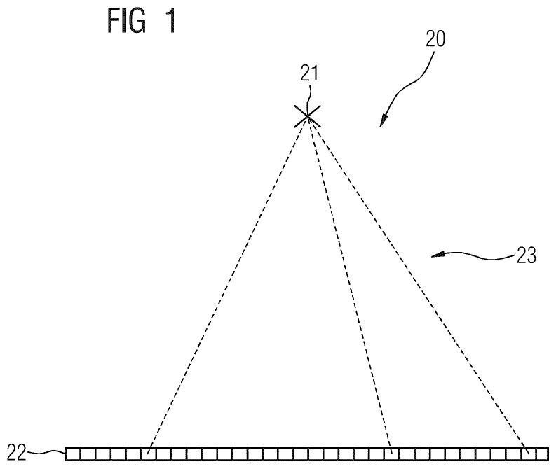

FIG. 1 shows a schematic diagram of an arrangement 20 for x-ray imaging.

The arrangement 20 has an x-ray radiation source 21 and an x-ray detector 22.

The x-ray radiation source 21 is arranged above the x-ray detector 22. An examination area 23 is provided between the x-ray radiation source 21 and the x-ray detector 22, in which an object to be examined can be placed, but in FIG. 1 is not placed there. The x-ray detector 22 is positioned below the examination area 23.

The x-ray radiation emitted from the x-ray radiation source 21 can pass through the examination area 23 to strike the x-ray detector 22. The path of the x-ray radiation from the x-ray radiation source 21 to the x-ray detector 22 is shown by dashed lines. The x-ray radiation of FIG. 1 is exclusively primary x-ray radiation because it is not scattered in an object. The trajectories of the x-ray photons are thus dead straight.

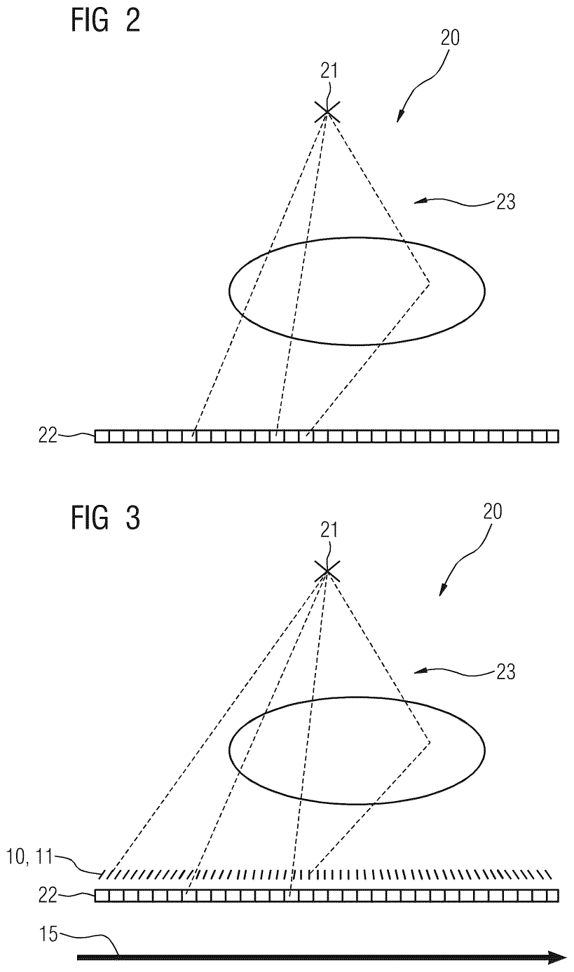

FIG. 2 shows a schematic diagram of an arrangement 20 for x-ray imaging with an object in the examination area 23.

The object is shown as an oval shape. Part of the x-ray radiation shown by a dashed line is scattered in the object. Along with the primary x-ray radiation, scattered x-ray radiation additionally strikes the x-ray detector 22. Scattered x-ray photons have a dashed line with a kink in FIG. 2.

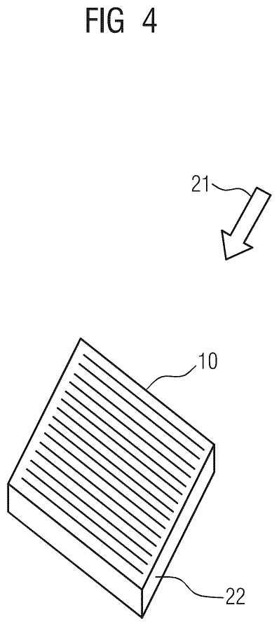

FIG. 3 shows a schematic diagram of an inventive arrangement 20 for x-ray imaging with a scattered radiation mask 10.

The inventive arrangement 20 has the x-ray radiation source 21, the examination area 23, a scattered radiation mask 10 and the x-ray detector 22. The examination area 23 is between the x-ray radiation source 21 and the x-ray detector 22. The scattered radiation mask 10 is arranged between the examination area 23 and the x-ray detector 22 and is aligned in such a way that the stack direction 15 of the stack of slices 11 is parallel to the x-ray detector 22. Arranged below the examination area 23 is the scattered radiation mask 10. Arranged on the lower part of the diagram is the x-ray detector 22, which is positioned so that in particular it can detect the primary x-ray radiation that passes through the examination area 23 and the scattered radiation mask 10. A cross section of at least one layer in the width direction is embodied in a trapezoidal shape.

FIG. 4 shows a perspective view of an inventive scattered radiation mask 10 for reduction of scattered x-ray radiation.

The scattered radiation mask 10 of FIG. 4 has a strip form. Indicated below the scattered radiation mask 10 is the x-ray detector 22. Indicated by an arrow above the scattered radiation mask 10 is the x-ray radiation source 21.

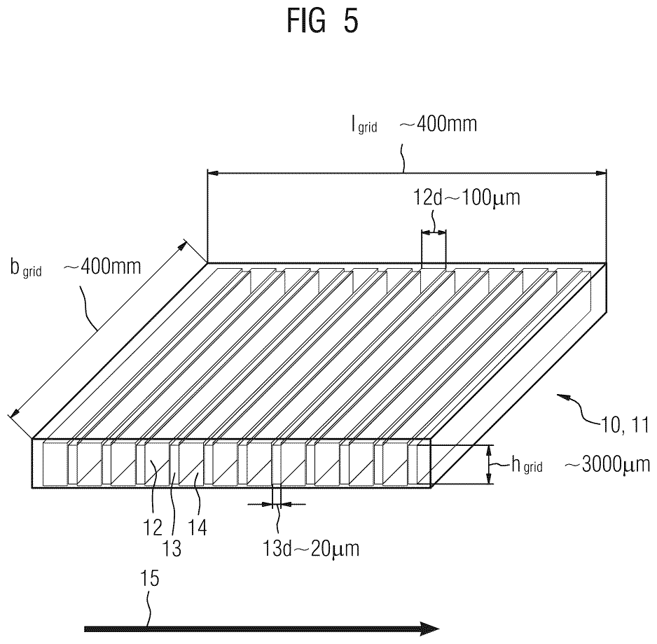

FIG. 5 shows a perspective detailed view of an inventive scattered radiation mask 10 for reduction of scattered x-ray radiation.

The scattered radiation mask 10 has a stack of slices 11. The stack of slices 11 has an x-ray radiation-transparent first slice 12, an x-ray radiation-absorbing second slice 13 and an x-ray radiation-transparent third slice 14. The first slice 12, the second slice 13 and the third slice 14 each have at least one planar layer. Neighboring layers of the stack of slices 11 can be applied to each other in a planar manner in the stack direction 15 via an additive manufacturing method.

The overall dimensions of the scattered radiation mask 10 in FIG. 5 amount to around 400 mm in length lgrid and 400 mm in breadth bgrid, with a total height hgrid of around 3000 μm. The overall dimension of the scattered radiation masks 10 can differ greatly from the values specified and are primarily limited upwards or downwards by the characteristics of the system for additive production. Advantageously one or more example embodiments of the present invention offer the possibility of producing scattered radiation grids in any size, depending on the size of the x-ray detector.

The x-ray radiation-transparent slices 12, 14 in FIG. 5 have a thickness 12d of around 100 μm, while the x-ray radiation-absorbing slice 13 is thinner, with a thickness 13d of around 20 μm. Such slices can be arranged in an alternating pattern over the entire length of the scattered radiation mask 10. FIG. 5 thus further shows that a thickness 13d of the second slice 13 amounts to less than 200 μm, in particular to less than 100 μm. In this exemplary embodiment a minimum structure size within a layer can be larger than a minimum thickness of this layer.

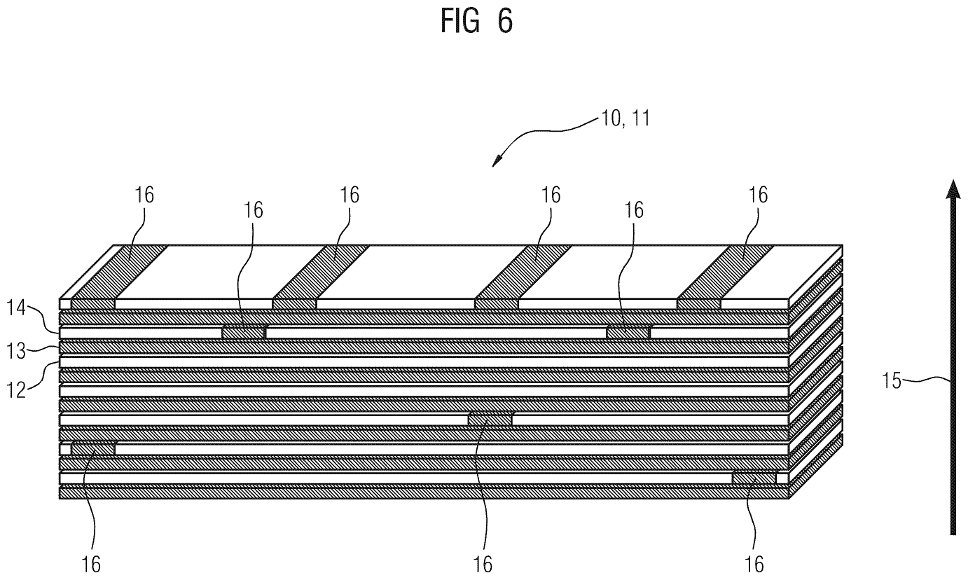

FIG. 6 shows a perspective detailed view of a first exemplary embodiment of the scattered radiation mask 10.

At least one layer of the first slice 12 and/or the third slice 14 in the width direction has an x-ray radiation-absorbing strip 16. FIG. 6 shows that x-ray radiation-absorbing strips 16 are arranged in a few of the x-ray radiation-transparent slices. These strips 16 can be positioned at regular intervals along the length of the respective layers. The x-ray radiation-absorbing strips 16 can occur in various layers of the stack of slices 11 and form a pattern that extends both horizontally and also vertically through the structure. In accordance with an advantageous development, the x-ray radiation-absorbing strips 16 of neighboring layers can be combined into an N-cornered structure, with N not equal to 4.

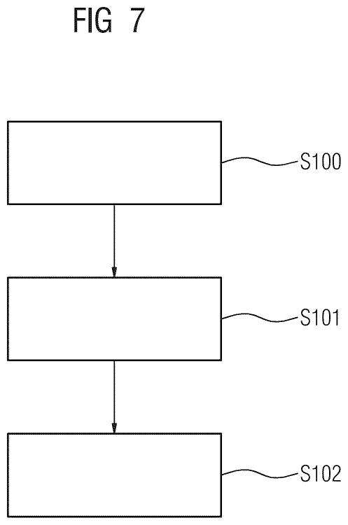

FIG. 7 shows a flow diagram of an inventive method with the steps S100 to S102. The method or individual steps or all method steps S100 to S102 shown in FIG. 7 can be carried out repeatedly in order to create a stack of slices 11 with a number of x-ray radiation-transparent slices and a number of x-ray radiation-absorbing slices, and in this way manufacture a scattered radiation mask 10 with the desired overall length lgrid.

Method steps S100 to S102 identify an application, via an additive manufacturing method, of layers in a planar manner onto one another in the stack direction to form a stack of slices in such a way that,
the stack of slices 11 has an x-ray radiation-transparent first slice 12, an x-ray radiation-absorbing second slice 13 and an x-ray radiation-transparent third slice 14 and
the first slice 12, the second slice 13 and the third slice 14 each have at least one planar layer. In one or each of the method steps S100 to S102 a thickness of a layer of the stack of slices 11 can basically be varied via the additive manufacturing method in the width direction, in particular in order to embody a cross section of this layer in the width direction in a trapezoidal shape.

In particular, method step S100 shows an application, via an additive manufacturing method, of at least one x-ray radiation-transparent layer of the first slice 12. In particular, method step S101 shows an application, via an additive manufacturing method, of at least one x-ray radiation-absorbing layer of the second slice 13 in a planar manner onto the at least one layer of the first slice 12 to form a stack of slices. In particular, method step S103 shows an application, via an additive manufacturing method, of at least of one x-ray radiation-absorbing layer of the third slice 14 in a planar manner onto the at least one layer of the second slice 13 to form a stack of slices. This exemplary embodiment thus in particular shows the possibility that the second slice 13 has a maximum of one layer and/or that the first slice 12 and optionally the third slice 14 each have a maximum of one layer.

The x-ray radiation-transparent slices 12, 14 are or will be embodied from a first material composition and the x-ray radiation-absorbing slice 13 is or will be embodied from a second material composition, wherein the first material composition and the second material composition are embodied differently from one another as regards a proportion of a material. The first material composition can have hollow glass spheres and/or an aerogel. As an alternative or in addition, the second material composition can have lead, tantalum, tungsten, rhenium, osmium, iridium, bismuth, platinum, thallium, mercury or gold. It is conceivable for the first material composition to have a plastic matrix and the second material composition to have the same plastic matrix.

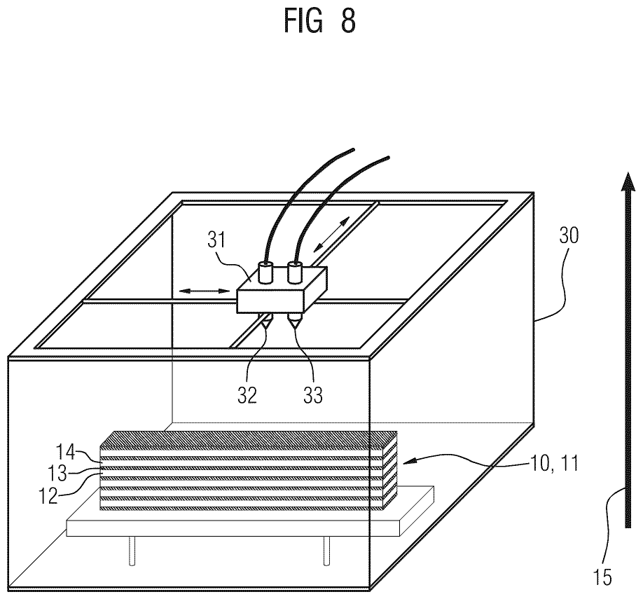

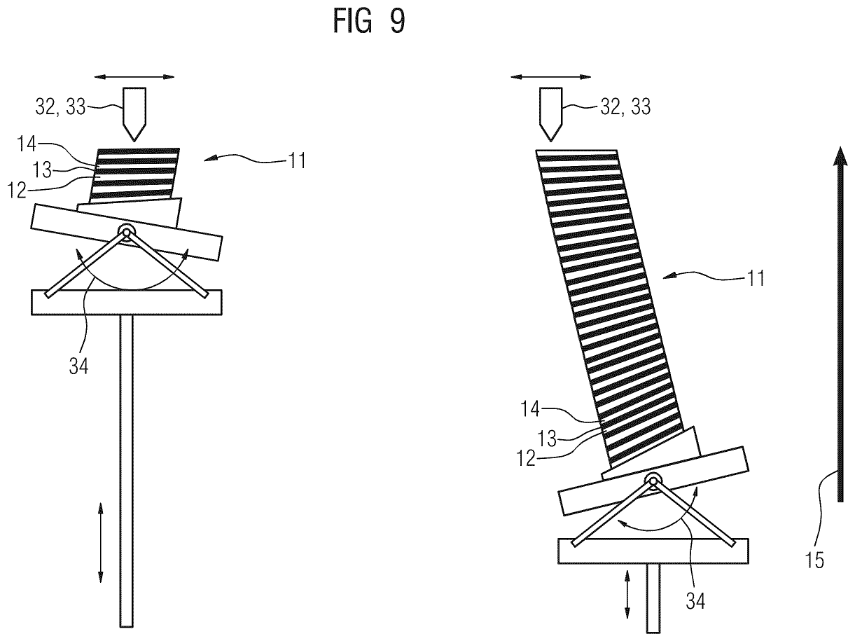

FIG. 8 shows a perspective view of an inventive system 30 for additive manufacturing of a scattered radiation mask 10.

The system 30 has an application unit 31, which is embodied to carry out an inventive manufacturing method for manufacturing the scattered radiation mask 10. The system 30 can have a number of components, which can be arranged in a vertical configuration. Arranged on the lower part of the system 30 can be a plate, as shown in FIG. 8, which can carry the stack of slices 11 and in particular can move vertically. This can make possible an adaptation of the distance between the application unit 31 and the stack of slices 11 when layers are applied. As shown in FIG. 8 a frame structure can be arranged above the stack of slices 11. This frame structure can be positioned parallel to the plate and be moveable in a horizontal plane, which is indicated by a double-ended arrow. The application unit 31 can be mounted on the frame structure. The system 30 of FIG. 8 is in particular embodied as a “core XY” arrangement, which can make possible an efficient additive production.

The application unit 31 of FIG. 8 has two application nozzles 32, 33, which can be used for application of x-ray radiation-transparent material and x-ray radiation-absorbing material for forming the slices 12, 13, 14 of the scattered radiation mask 10. The application unit 31 can be moveable along the frame structure, which can make possible the application of material over the entire surface of the stack of slices 11 lying below it. The application unit 31 can be connected via a number of cables or pipes. These can serve to supply the application unit 31 with materials, energy or control signals. The application nozzles 32, 33 can each have an oval or rectangular cross section.

FIG. 9 shows a schematic side view of a first exemplary embodiment of the system. Contained in FIG. 9 are two configurations of the system 30 for two different points in time of the manufacturing of the stack of slices 11.

The system 30 of FIG. 9 has a tilt apparatus 34 below the stack of slices 11. The tilt apparatus 34 is embodied to set the tilt angle of the stack of slices 11 relative to the application nozzles 32, 33. The tilt apparatus 34 is in particular embodied to change the tilt angle of the application unit 31 relative to a layer already applied.

In the left-hand configuration the plate on which the stack of slices 11 is applied layer by layer is shown tilted to the right. The application nozzles 32, 33 are embodied to apply a layer to a layer already produced in a planar manner. In the right-hand configuration the tilt apparatus 34 has adapted the angle of the stack of slices 11 in such a way that the plate is tilted to the left. The tilt apparatus 34 can in particular set a different tilt angle for each layer. The tilt angle typically varies gradually from layer to layer in the stack direction 15.

The application nozzles 32, 33 are shown by horizontal arrows, which indicates their sideways movement during the application process. The stack of slices 11, in both configurations, shows alternating bright and dark slices, which can represent the x-ray radiation-transparent slices 12, 14 and x-ray radiation-absorbing slices 13.

The construction of the system 30 can make possible the manufacturing of focused scattered radiation masks 10, in that the angle of application can be set by the tilt apparatus 34. This can make possible the production of masks with varying geometries in order to fulfill various requirements of x-ray imaging. This form of embodiment is in particular advantageous when a thickness of a layer or of each layer of the stack of slices 11 is varied in the width direction via the additive manufacturing method. This in particular enables a focused scattered radiation mask to be produced, in which a cross section of a layer or of each layer is embodied trapezoidal in the width direction.

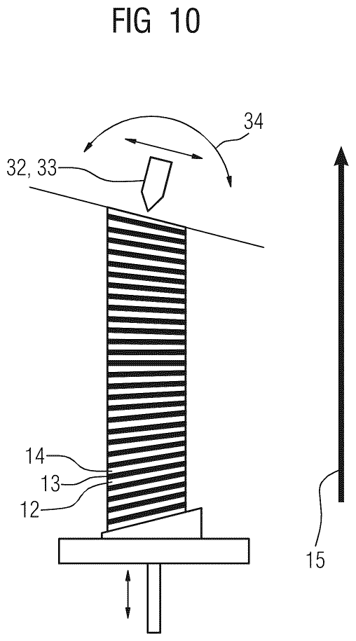

FIG. 10 shows a schematic side view of a second exemplary embodiment of the system 30. The exemplary embodiment of FIG. 10 can be an alternative or addition to the exemplary embodiment of FIG. 9.

The system 30 of FIG. 10 has a tilt apparatus 34, which is embodied to change a tilt angle of the application unit 31 by the tilt apparatus 34 relative to a layer already applied. In this exemplary embodiment the tilt apparatus 34 is arranged for example between the frame structure and the application unit 31, so that the application unit 31, in particular the two application nozzles 32, 33, are able to be tilted in relation to the stack direction 15. By comparison, the application nozzles 32, 33 of FIG. 9 are not able to be tilted in relation to the stack direction 15, but the plate with the stack of slices 11 is able to be tilted in relation to the stack direction 15.

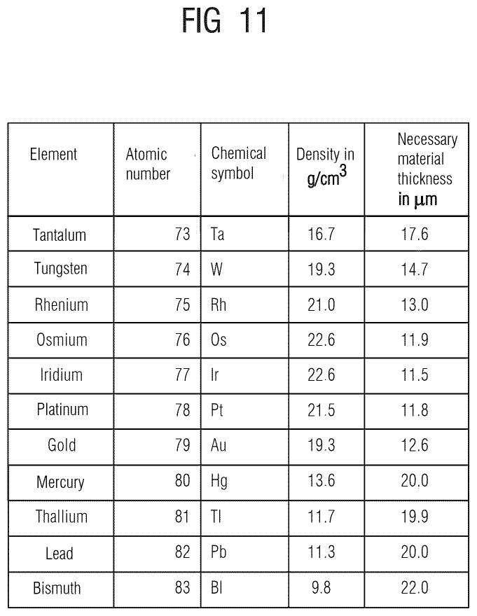

FIG. 11 shows a table with materials for the x-ray radiation-absorbing layers. The elements listed in the table can be used for the x-ray radiation-absorbing second layer of the scattered radiation mask. The material composition of the second layer can feature lead, tantalum, tungsten, rhenium, osmium, iridium, bismuth, platinum, thallium, mercury or gold.

The table in FIG. 11 has five columns: Element, atomic number, chemical symbol, density in g/cm3 and necessary material thickness in μm in relation to lead. The atomic numbers extend from 73 for tantalum to 83 for bismuth. The densities extend from 9.8 g/cm3 for bismuth to 22.6 g/cm³ for osmium and also iridium. The necessary material thickness compared to lead with 20.0 μm extends from 11.5 μm for iridium to 22.5 μm for bismuth.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

Independent of the grammatical term usage, individuals with male, female or other gender identities are included within the term.

Although the present invention has been illustrated and described in greater detail by the preferred exemplary embodiments, the present invention is not restricted by the disclosed examples and other variations can be derived herefrom by the person skilled in the art, without departing from the scope of protection of the present invention.

What is claimed is:

1. A scattered radiation mask for reduction of scattered x-ray radiation, the scattered radiation mask comprising:
a stack of slices including an x-ray radiation-transparent first slice, an x-ray radiation-absorbing second slice and an x-ray radiation-transparent third slice, wherein
each of the x-ray radiation-transparent first slice, the x-ray radiation-absorbing second slice and the x-ray radiation-transparent third slice have at least one planar layer, and
neighboring layers of the stack of slices are applied to one another in a planar manner via an additive manufacturing method in a stack direction.

2. The scattered radiation mask as claimed in claim 1, wherein a thickness of the x-ray radiation-absorbing second slice is less than 200 μm.

3. The scattered radiation mask as claimed in claim 1, wherein
the x-ray radiation-transparent first slice has a first material composition,
the x-ray radiation-absorbing second slice has a second material composition, and
the first material composition and the second material composition are different from one another with regard to a proportion of a material.

4. The scattered radiation mask as claimed in claim 3, wherein the first material composition includes at least one of hollow glass spheres or an aerogel.

5. The scattered radiation mask as claimed in claim 3, wherein the second material composition includes lead, tantalum, tungsten, rhenium, osmium, iridium, bismuth, platinum, thallium, mercury or gold.

6. The scattered radiation mask as claimed in claim 3, wherein the first material composition includes a plastic matrix and the second material composition includes a same plastic matrix.

7. The scattered radiation mask as claimed in claim 1, wherein at least one of the x-ray radiation-transparent first slice or the x-ray radiation-absorbing second slice has a maximum of one layer.

8. The scattered radiation mask as claimed in claim 1, wherein a layer of at least one of the x-ray radiation-transparent first slice or the x-ray radiation-transparent third slice has an x-ray radiation-absorbing strip in a width direction.

9. The scattered radiation mask as claimed in claim 8, wherein x-ray radiation-absorbing strips of neighboring layers combine to form an N-cornered structure, wherein N is not equal to 4.

10. The scattered radiation mask as claimed in claim 1, wherein a minimum structure size within a layer is greater than a minimum thickness of the layer.

11. The scattered radiation mask as claimed in claim 1, wherein a cross section of a layer in a width direction has a trapezoidal shape.

12. An arrangement for x-ray imaging, the arrangement comprising:
the scattered radiation mask as claimed in claim 1;
an x-ray radiation source;
an x-ray detector; and
an examination area between the x-ray radiation source and the x-ray detector; wherein
the scattered radiation mask is between the examination area and the x-ray detector, and
the scattered radiation mask is aligned such that the stack direction of the stack of slices is parallel to the x-ray detector.

13. A method for manufacturing a scattered radiation mask for reduction of scattered x-ray radiation, the method comprising:
applying, via an additive manufacturing method, layers onto one another in a planar manner and in a stack direction to form a stack of slices, wherein
the stack of slices has an x-ray radiation-transparent first slice, an x-ray radiation-absorbing second slice and an x-ray radiation-transparent third slice, and
each of the x-ray radiation-transparent first slice, the x-ray radiation-absorbing second slice and the x-ray radiation-transparent third slice has at least one planar layer.

14. The method as claimed in claim 13, wherein a thickness of a layer of the stack of slices is varied in a width direction via the additive manufacturing method.

15. A system for additive manufacturing of a scattered radiation mask for reduction of scattered x-ray radiation, the system comprising
an application unit configured to carry out the method as claimed in claim 13.

16. The system as claimed in claim 15, wherein the application unit includes at least one application nozzle with an oval or rectangular cross section.

17. The system as claimed in claim 15, wherein
the system includes a tilt apparatus, and
the application unit and the tilt apparatus are arranged such that a tilt angle of the application unit is arranged variably relative to a layer already applied by the tilt apparatus.

18. A method comprising:
using a system including an application unit for additive manufacturing of the scattered radiation mask as claimed in claim 1.

19. The scattered radiation mask as claimed in claim 1, wherein the scattered radiation mask is configured to reduce scattered x-ray radiation for x-ray imaging.

20. The scattered radiation mask as claimed in claim 2, wherein the thickness of the x-ray radiation-absorbing second slice is less than 100 μm.

21. The scattered radiation mask as claimed in claim 7, wherein the x-ray radiation-transparent third slice has a maximum of one layer.
